(19) United States
(12) Patent Application Publication
(10) Pub. No.: US 2010/0262146 A1
(43) Pub. Date: Oct. 14, 2010

(54) Disposable bone cutting instrument

(75) Inventor: Peter Tulkis, Paramus, NJ (US)

Correspondence Address: LERNER, DAVID, LITTENBERG,;KRUMHOLZ & MENTLIK, 600 SOUTH AVENUE WEST, WESTFIELD, NJ, 07090, US

(73) Assignee: Howmedica Osteonics Corp., Mahwah, NJ (US)

(21) Appl. No.: 12/384,900

(22) Filed: Apr. 9, 2009

(51) Int. Cl. A61B 17/00; B21K 5/04

(52) U.S. Cl. 606/80; 76/108.1

(57) ABSTRACT

A helical reamer for reaming a bone cavity has a longitudinally extending support shaft having a first driven end and a second end having a tip for insertion into the bone cavity. The research for a multiplicity of stamped flat reamer plates each having a plurality of cutting flutes formed on an external surface thereof and a central internal opening to receive the support shaft. The internal opening includes a coupling element in the form of a key and keyway rotationally coupling each reamer plate to the support shaft. The key and keyway allowing each reamer plate to be slightly rotated with respect to the flutes of an adjacent reamer plate. The coupling elements for rotationally coupling the plates to the drive shaft can be mating flattened areas on the internal surface of the stamping and the external surface of the drive shaft. A broach of stamped plates is also shown.

### BACKGROUND OF THE INVENTION

The present invention relates to the field of instruments for the implantation of prostheses, and more specifically to reamers, for forming the inside cavity of a bone for receipt of a complimentary prosthesis. More particularly, to an orthopedic reamer that has been designed to permit the use of low-cost manufacturing methods while being durable and still maintaining its ability to create such canals.

The replacement of joints, most frequently hips, in persons having damaged, diseased or malformed joints has become more and more frequent. Since hip replacement is the most common of such operations, the same will be described herein as exemplary of use of the present invention. Such instruments could be used in humeral cavity preparation as well.

In the case of hip replacements, some operations involve the replacement of the ball or proximal end of the femur with a suitable prosthesis, while other such operations also include the installation of a socket or acetabular prosthesis. In either event however, the proximal femur is prepared for receipt of the femoral component by reaming a generally cylindrical hole or conical hole (slightly tapered) into the femur from the proximal end thereof to the diaphysis, and then, if necessary, appropriately broaching or rasping out substantially all of the remaining cancellous bone so as to shape the same to be complimentary with both the neighboring cortical bone and the adjacent portion of the prosthesis to be installed therein. In many cases, a prosthesis of a standard size broaches may be used. Even in these cases however, it is common to provide and use multiple progressively larger reamers which increase in diameter in ½ to 1 mm increments and more than one broach size, such as by way of example, a slightly undersized broach for rough forming of the bone opening, and a nominal size for finishing the cavity. Given the need for different size reamers and broaches for a given implant, and the various size implants available, standard reamers and broaches are now available in a broad range of sizes.

In the prior art, bone reamers and broaches were generally fabricated from a solid piece of metal. Since the prosthesis must in effect be supported by the inside of the bone, and the inner surface of the bone at the proximal end thereof may be a complicated three-dimensional contour, the machining of a broach to accurately match this three dimensional contour requires special equipment for progressively generating the cutting edges which will define the desired three dimensional contour.

It is also one aspect of the present invention to reduce the cost of such reamers and broaches by simplifying the manufacturing procedure to achieve, in a much less costly manner, bone reamers and broaches for the desired contours and characteristic manufactured of appropriate materials. Another aspect of the invention is to achieve a better cutting action by allowing more flexibility in the instrument cutting edge design. Still another aspect is to provide a reamer broach which may be more easily and accurately inspected to verify the cutting contours at various positions thereof.

Typically, an orthopedic reamer, or set of reamers of varying diameters and lengths, is utilized to increase the diameter of the canal in a bone. This is generally done on long bones within the body, but can be done on any bone suitable for the remaining process. Normally, the canal is progressively reamed in 0.5, 1 or 2 mm increments until the desired diameter is reached. Examples of reamers of this type are shown in U.S. Pat. No. 6,168,599 to Frieze et al. and U.S. Pat. No. 6,162,226 to DeCarlo, Jr. et al. A broach is shown in U.S. Pat. No. 5,006,121 and a disposable reamer is shown in U.S. Patent Publication No. 2006/0004371.

While orthopedic reamers such as those described above are capable of creating or increasing the diameter of canals in a bone, they have their shortcomings. Most importantly, the manufacturing costs associated with orthopedic reamers have traditionally been high. A standard reamer is typically constructed of a metallic or other hard material machined from a solid block or rod or from several solid pieces that are assembled to form the reamer. These high costs have required such reamers to be utilized in multiple procedures. This re-use requires the cleaning and sterilization of such a reamer before each use, which adds significant additional costs. Improper cleaning and sterilization can lead to disease transmission. Furthermore, multiple uses of a reamer create the greater probability of failure due to fatigue and/or poor cutting due to wear of the cutting surfaces of the reamer. Hence, a disposable single use inexpensive orthopedic reamer would be advantageous.

For the foregoing reasons, there is a need for a reamer that can be inexpensively manufactured and suitable for single use, while maintaining the required precise and accurate dimensions needed for reaming a bone.

### SUMMARY OF THE INVENTION

These and other aspects of the invention are provided by a helical reamer for reaming a bone cavity having a longitudinally extending support shaft having a first driven end and a second end having a tip for insertion into the bone cavity. A multiplicity of flat reamer plates are provided each having a plurality of cutting flutes formed on an external surface thereof and a central internal opening to receive the support shaft. The internal opening has a coupling element rotationally coupling each reamer plate to the support shaft. The coupling element allows each reamer plate to be slightly rotated with respect to the flutes of an adjacent reamer plate. At least some of the reamer plates have a different external cross-section. Preferably the reamer plates have external cross-sections which progressively increase on moving from the second end of the support shaft towards the first end of the support shaft. In one embodiment each plate internal opening includes an inwardly extending tab for engaging a groove on an outer surface of the support shaft, the groove extending between the first and second ends of the shaft. The groove may extend helically around an outer surface of the shaft or the groove may extend parallel to a rotational axis of the support shaft. Alternatively internal opening of each plate is part circular with a flat portion along one side of the opening and the support shaft has a corresponding flat portion along an outer surface thereof for mating with the flat portion on the plate. The flat portion of the shaft extends helically around the outer surface of the support shaft between the first and second ends of the shaft.

In the reamer each plate has flutes having a leading surface of a first length and at a first angle and a trailing edge of a second length and a second angle with the first length being greater than the second length and the first angle being stepper than the second angle. In a conical reamer or broach the external cross-sections of the plates decrease on moving from the first driven end to the second end to form a generally conical outer reamer surface. The outer surface of each plate may taper inwardly towards a longitudinal axis of the support shaft. The inward taper on each plate equals the angle of the generally conical reaming surface.

The method for producing a bone reamer is also disclosed and comprises stamping a multiplicity of different size plates from sheet metal, each plate having at least two flutes having a leading cutting surface and a trailing surface forming a pocket between the adjacent flutes. Each plate has an internal opening with a key portion. A longitudinally extending drive shaft is inserted within the internal opening of each plate of the multiplicity of plates. The drive shaft has an external key portion for mating with the key portion of the internal opening. The multiplicity of plates are fixed on the drive shaft in the longitudinal direction. In one embodiment the key portions are mating flattened areas on the internal surface of the stamping and the external surface of the drive shaft. Alternately the key portion on the stamping and shaft may be a projection and a corresponding groove.

For ease of manufacture the multiplicity of different size plates are formed on a progressive die. Alternately the plates may be bonded to the shaft or the shaft may be a polymeric material injection molded into the internal opening. In addition the plates may be clamped onto the drive shaft by a nut at a leading end thereof.

### BRIEF DESCRIPTION OF THE DRAWINGS

FIG. 1 is an isometric view of the reamer of the present invention including a metal shaft on which the cutting elements are mounted;

FIG. 2 is an isometric view of the reamer shown in FIG. 1 with the cutting portion removed from the drive shaft;

FIG. 3 is an exploded view of the cutting element portion of the reamer shown in FIG. 2;

FIG. 4 is a plan view of three progressively larger slices or plates of the reamer cutting element portion shown in FIG. 3;

FIG. 4A is a stack of three discs helically offset from the adjacent disc;

FIG. 5 is a partial view of the cutting portion of the reamer as shown in FIG. 3 having a tapered conical cross-section;

FIG. 6 is a cross-sectional view of a broach made by the manufacturing process of the present invention;

FIGS. 7A-7D are a top, bottom, elevation and isometric view of two slices or plates of the broach shown in FIG. 6.

FIG. 8 is a stack of three slices or plates showing the draft angle or taper angle of the plates; and

FIG. 9 is a stack of three slices or plates enlarged in steps.

### DETAILED DESCRIPTION

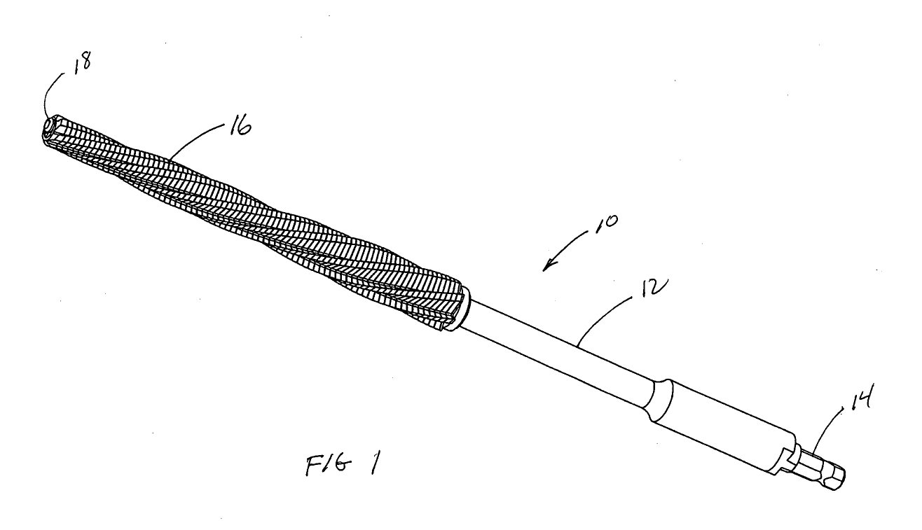

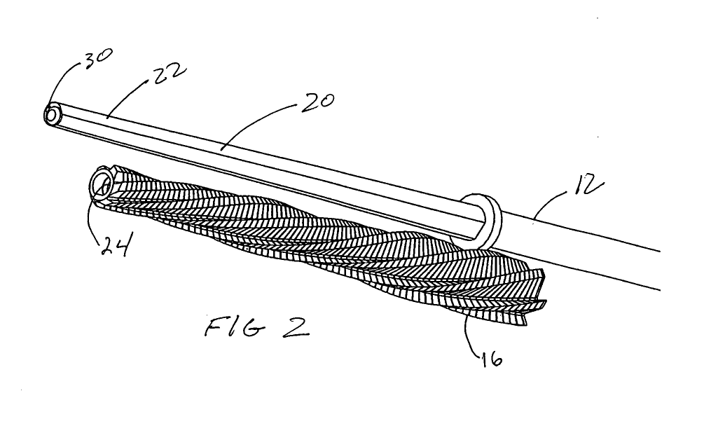

Referring to FIG. 1 there is shown an isometric view of a reamer generally denoted as 10 including a shaft 12 with a drive end 14 and a leading cutting end 16 including a leading tip 18. Drive shaft 12 may be either made of metal or plastic. Referring to FIG. 2 there is shown the reamer of FIG. 1 with the cutting portion 16 removed from a leading end 20 of drive shaft 12. In the preferred embodiment the leading shaft 20 of drive shaft 12 includes a flattened portion 22 shaped to engage a flattened portion 24 located within the hollow interior 26 of cutting portion 16. In the preferred embodiment the flattened portion 22 of shaft portion 20 is surrounded in part by a portion 28 rotatably coupled to portion 22 by a key 30.

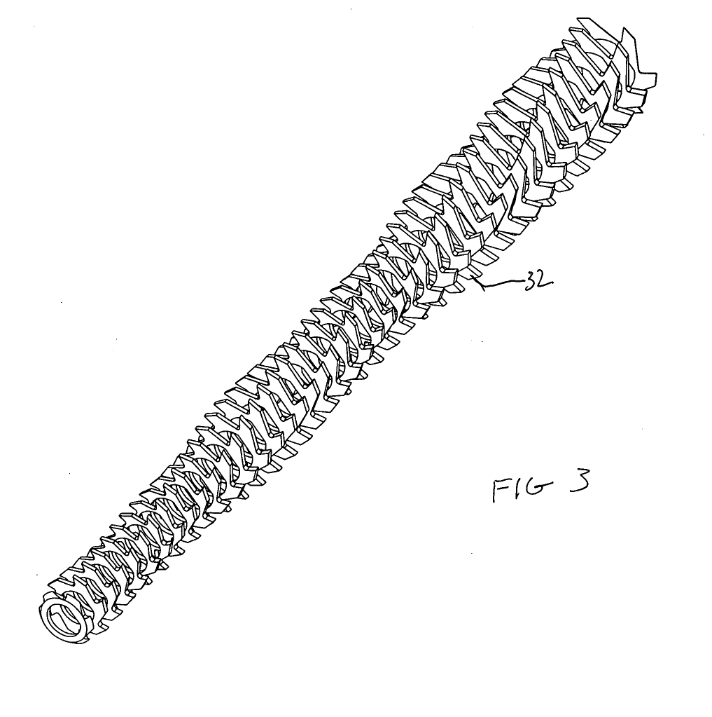

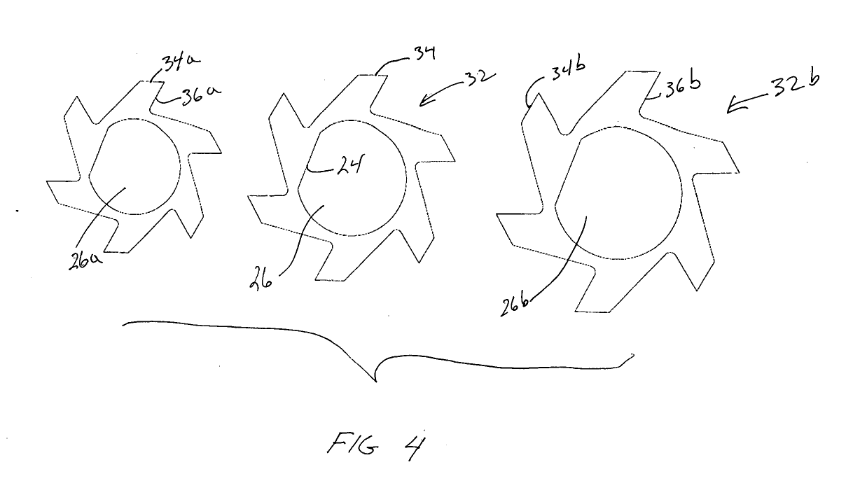

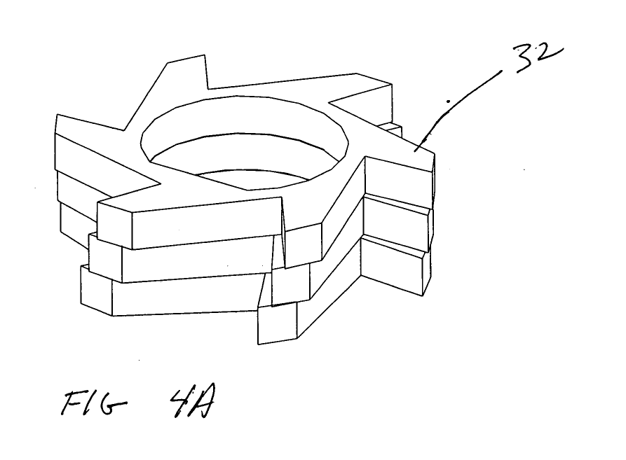

As best shown in FIG. 3, cutting portion 16 is composed of a multiplicity of individual slices or plates 32. As can be seen in both FIGS. 2 and 3, plates 32 preferably are larger at an end of the cutter adjacent the drive end 14 of shaft 12 and gradually becomes smaller toward the leading end or tip 18 of the remaining instrument. This shape is dictated by the design of the femoral component to be implanted in the proximal femur which typically tapers outwardly in a distal to proximal direction. As can be seen in FIG. 4, each plate 32 includes an outwardly extending cutting flute 34 having a leading cutting edge 36 for reaming the medullary canal of the femur. Typically four or more flutes are included on each slice 32 although as the slices or plates reduce in size fewer cutting elements may be included. Each flute 34 has a trailing edge 38 and a pocket is thereby formed between the trailing edge of one flute and the leading cutting edge of the next adjacent flute. This pocket 40 allows for the capture of bone chips formed during the cutting operation.

In the preferred embodiment, the flat 24 within the hollow interior 26 of each plate or slice 32 shifts slightly so that the pockets 40 of adjacent plates 32 form a helix extending in the longitudinal direction of the drive shaft 12. The helical flute design is typical in bone reamers and provides a path for the bone chips developed during reaming to exit the bore in the femur. The plates are interlocked together during the assembly in progressive die. The entire stack of plates forms the cutting geometry of the reamer. The stack is inserted over the reamer shaft and it is captured at the bottom by the spring-loaded locking tab or pin. The torque is transferred from external geometry of the shaft to the internal geometry of cutting stack.

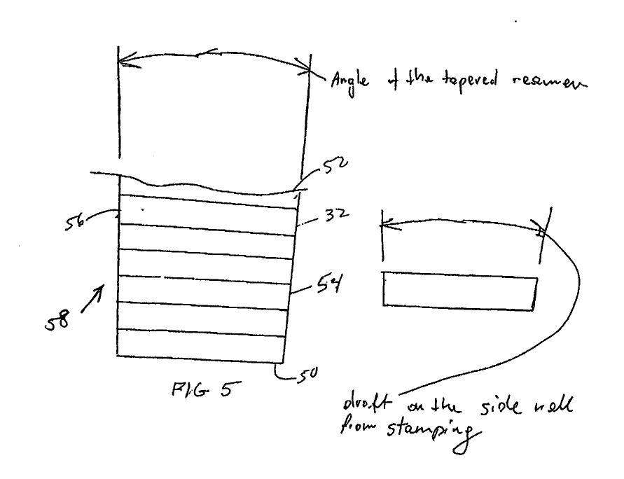

Referring to FIG. 5, there is shown a group of plates or discs 32 forming a conical reamer. The reamer has a taper angle of 3 to 7 degrees and, as indicated above, increases in cross-sectional extent on moving from a distal end 50 to a proximal end 52. The outer surface 54 of the conical reamer can be made smooth if the draft angle i.e. the angle formed on the edge 56 of each plate or disc 32 which edge is formed on the plate 32 during the preferred manufacturing method is equal to the angle of the tapered reamer. Thus, the sidewall of each plate would extend at an angle of 5 to 7 degrees matching that of the conical reamer.

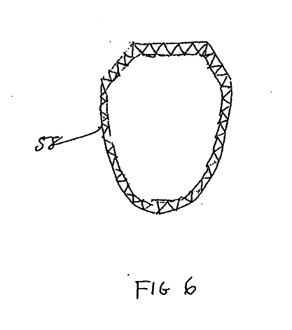

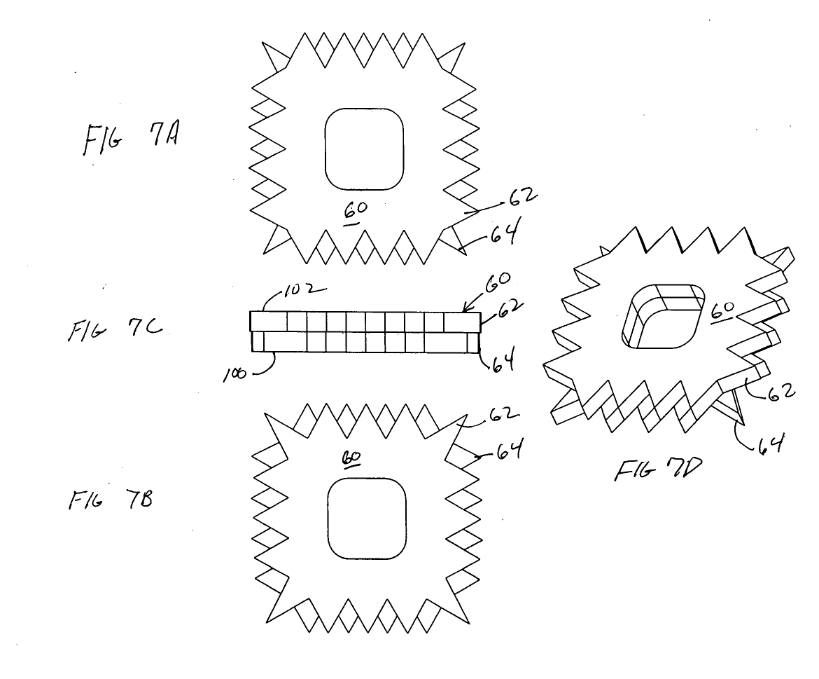

Referring to FIGS. 6 and 7A to 7D, there is shown a broach 58 made up of a plurality of stamped plates 60 each having a plurality of triangular teeth 62, 64 which teeth are offset from one another when the plates are assembled vertically. The tips 66 of each tooth 62, 64 form a cutting operation on the bone. Like the reamer 10 described above, broach 58 is preferably conically tapered so that each plate 60 increases in cross-sectional moving in a distal to proximal direction.

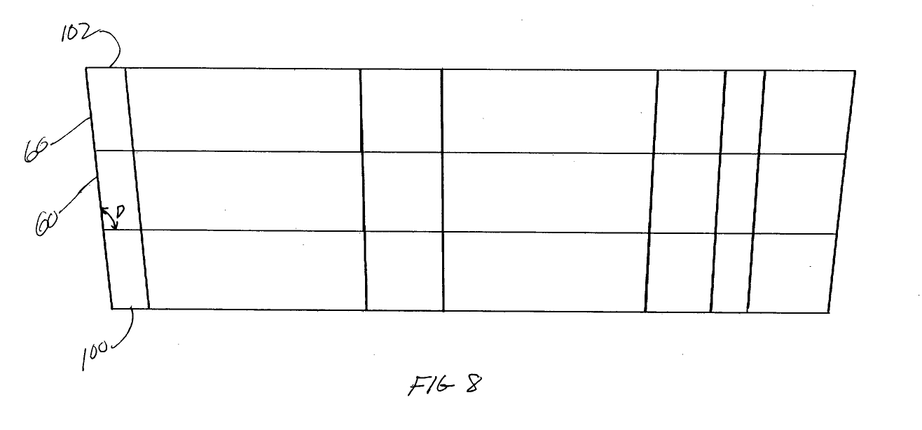

Referring to FIG. 8 there is shown three discs or plates 60 with each disc having the same draft angle D for teeth 62, 64. Thus the bottom surface 100 of each disc is smaller than a top surface 102. However, the top surface of this lower disc is equal to the bottom surface of the upper disc. It may be 3°-7°.

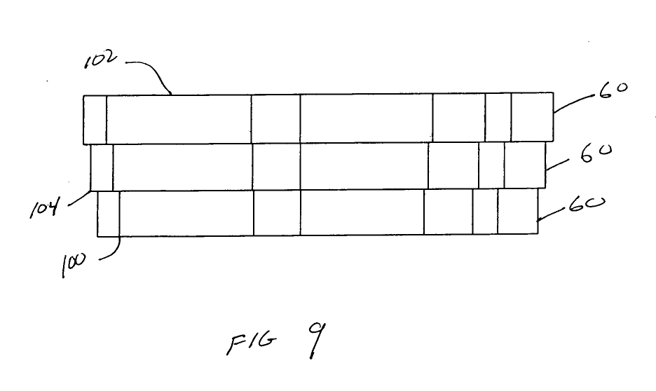

As shown in FIGS. 7A and 9 alternately the broach can have a stepped form where sidewalls of the discs are perpendicular with respect to the top and bottom discs. In this situation the tope of a disc will be smaller than the bottom of the adjacent disc above it assuming the broach 58 is tapered. These designs can also be used with the discs 32 of reamer 10.

The preferred manufacturing method for the disposable, low cost femoral cutting instruments, such as femoral reamers 10 and femoral broaches 58 of the present invention is by stamping. Instruments are preferably manufactured by utilization of sheet-metal lamination.

Femoral Reamers:

The cutting portion of a femoral reamer 10 is preferably constructed from flat geometries punched out from sheet metal by a progressive die. A number of discs are assembled vertically one to another on the same central axis. Each disc has internal and external geometries. The internal geometry consists of a circular opening for the driver shaft with some anti-rotational feature such as: flat, tab or recessed geometry matching the geometry of the shaft in a way making it possible to transfer torque from the shaft to the slice. The external geometry of the slice allows for bone removal. The outside geometry is consistent with the cross-section of the typical cutting tool having multiple cutting edges and back-side reliefs. The variety of desired configurations could be achieved by manipulation of the internal and external geometries. To construct a cylindrical reamer of certain diameter with the straight flute configuration requires the assembly of identical geometry or shaped discs. To construct a cylindrical reamer with the helical sweep or reverse helical sweep edge the outside geometry or shape would be identical from slice to slice, but internal geometry would be slightly rotated around the central axis from disc to disc to achieve the desired edge shift from slice to slice when assembled on the straight driver shaft. To build conical reamer 10, each disc would have similar geometry but would increase in size from slice to slice, when vertically assembled. It could have straight or helical flute geometry as described above. The conical geometry would have a grid or profile based upon the thickness of slices and the increase of the size of the cutting geometry from slice to slice. Steps could be eliminated by achieving a slight draft on the side surfaces of each slice during the punching steps. If the draft equaled the angle of the cone on the reamer, the smooth edge and surface angular transition can be achieved for entire cutting member.

Each slice would be interlocked with the neighboring slices via typical methods employed in the progressive die industry. The cutting member will come out of the die assembled, having certain degree of integrity. This will allow further assembly with a bulk part rather than single slices.

Three assembly configurations are possible for a stacked cutting member:

1—A metal shaft with a spring-loaded locking feature, such as a pin, to retain cutting elements on the shaft;

2—A plastic shaft permanently attached to the cutting member via ultrasonic weld; and

3—A plastic shaft insert molded into the cutting member.

The femoral broach 58 is manufactured in the similar fashion. It may be constructed from punched-out slices placed one on another in the vertical direction. In the preferred embodiment the teeth from slice to slice would be placed in the offset direction so the tooth from the bottom slice removes bone in one vertical plane and the tooth in the slice above removes bone in the vertical plane moved over in one direction by the distance of one half of the tooth. This would result from slice to slice, to ensure bone removal around the entire perimeter of the broach. Slices would be hollow on the inside. Plastic injection molding would fill the inside up to provide integrity for the construct.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A helical reamer for reaming a bone cavity comprising:
a longitudinally extending support shaft having a first driven end and a second end having a tip for insertion into the bone cavity;
a multiplicity of flat reamer plates each having a plurality of cutting flutes formed on an external surface thereof and a central internal opening to receive the support shaft, the internal opening including a coupling element rotationally coupling each reamer plate to the support shaft, the coupling element allowing each reamer plate to be slightly rotated with respect to the flutes of an adjacent reamer plate.

2. The reamer as set forth in claim 1 wherein at least some of the reamer plates have a different external cross-section.

3. The reamer as set forth in claim 2 wherein the reamer plates have external cross-sections which progressively increase on moving from the second end of the support shaft towards the first end of the support shaft.

4. The reamer as set forth in claim 1 wherein each plate internal opening includes an inwardly extending tab for engaging a groove on an outer surface of the support shaft, the groove extending between the first and second ends of the shaft.

5. The reamer as set forth in claim 4 wherein the groove extends helically around an outer surface of the shaft.

6. The reamer as set forth in claim 4 wherein the groove extends parallel to a rotational axis of the support shaft.

7. The reamer as set forth in claim 1 wherein internal opening of each plate is part circular with a flat portion along one side of the opening and the support shaft has a corresponding flat portion along an outer surface thereof for mating with the flat portion on the plate.

8. The reamer as set forth in claim 7 wherein the flat portion of the shaft extends helically around the outer surface of the support shaft between the first and second ends of the shaft.

9. The reamer as set forth in claim 1 wherein each plate has flutes having a leading surface of a first length and at a first angle and a trailing edge of a second length and a second angle with the first length being greater than the second length and the first angle being stepper than the second angle.

10. The reamer as set forth in claim 2 wherein the external cross-sections of the plates decrease on moving from the first driven end to the second end to form a generally conical outer reamer surface.

11. The reamer as set forth in claim 10 wherein an outer surface of each plate tapers inwardly towards a longitudinal axis of the support shaft.

12. The reamer as set forth in claim 11 wherein the inward taper on each plate equals the angle of the generally conical reaming surface.

13. A method for producing a bone reamer comprising:
stamping a multiplicity of different size plates from sheet metal, each plate having at least two flutes having a leading cutting surface and a trailing surface forming a pocket between the adjacent flutes, each plate having an internal opening with a key portion;
inserting a longitudinally extending drive shaft within the internal opening of each plate of the multiplicity of plates, the drive shaft having an external key portion for mating with the key portion of the internal opening; and
fixing the multiplicity of plates on the drive shaft in the longitudinal direction.

14. The method as set forth in claim 13 wherein the key portions are mating flattened areas on the internal surface of the stamping and the external surface of the drive shaft.

15. The method as set forth in claim 13 wherein the key portion on the stamping and shaft are a projection and a corresponding groove.

16. The method as set forth in claim 13 wherein the multiplicity of different size plates are formed on a progressive die.

17. The method as set forth in claim 13 wherein the plates are bonded to the shaft.

18. The method as set forth in claim 17 wherein the shaft is a polymeric material injection molded into the internal opening.

19. The method as set forth in claim 13 wherein the plates are clamped onto the drive shaft by a nut at a leading end thereof.
